SEQUENCE LISTING (2 sequences)

SEQ ID NO: 1 - protein, 153 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 2 - protein, 195 aa, Homo sapiens
MGKISSLPTQLFKCCFCDFLKVKMHTMSSSHLFYLALCLLTFTSSATAGPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGWPKTHPGGEQKEGTEASLQIRGKKKEQRREIGSRNAECRGKKGK

(19) United States
(12) Patent Application Publication
(10) Pub. No.: US 2009/0105141 A1
(43) Pub. Date: Apr. 23, 2009

(54) INTRAVENTRICULAR PROTEIN DELIVERY FOR AMYOTROPHIC LATERAL SCLEROSIS

(75) Inventors: James Dodge, Worcester, MA (US); Ronald K. Scheule, Hopkinton, MA (US)

Correspondence Address: GENZYME CORPORATION;LEGAL DEPARTMENT, 15 PLEASANT ST CONNECTOR, FRAMINGHAM, MA, 01701-9322, US

(73) Assignee: GENZYME CORPORATION, Cambridge, MA (US)

(21) Appl. No.: 12/175,870

(22) Filed: Jul. 18, 2008

Related U.S. Application Data
(63) Continuation of application No. PCT/US2007/001599, filed on Jan. 22, 2007.
(60) Provisional application No. 60/760,377, filed on Jan. 20, 2006.

(51) Int. Cl. A61K 38/18; A61P 25/00

(52) U.S. Cl. 514/12

(57) ABSTRACT

Amyotrophic Lateral Sclerosis can be successfully treated using intraventricular delivery of a neurotrophic growth factor, IGF-1. The administration can be performed slowly to achieve maximum effect. Effects are seen on both sides of the blood-brain barrier, making this a delivery means for Amyotrophic Lateral Sclerosis which affects both brain and skeletal muscle.

### TECHNICAL FIELD OF THE INVENTION

This invention is related to the area of Amyotrophic Lateral Sclerosis. In particular, it relates to the treatment and/or prevention of this disease by protein therapy.

### SUMMARY OF THE INVENTION

Amyotrophic Lateral Sclerosis (ALS) is a fatal disease in which motor neurons progressively degenerate in the spinal cord, brain stem, and cerebral cortex. Loss of upper motor neurons is responsible for loss of descending supraspinal innervation and loss of lower motor neurons is responsible for loss of innervation of skeletal muscle. Cognitive impairment is often observed. Symptoms of ALS include exertional/rest dyspnea, orthopnea, poor cough, constipation, low voice volume, poor quality sleep, morning headache, daytime sleepiness, apneas, choking spells, noisy breathing, coughing with eating, clumsiness, twitching, cramping, weakness, slurring of speech, difficulty with speech and swallowing, and pathological laughing or crying. ALS occurs more frequently in males than females, and the prevalence increases with age.

There are many types of ALS, including sporadic, familial, and Pacific. Among the familial ALS sufferers, about ¼ contain a point mutation in the SOD gene, i.e., the gene encoding Cu/Zn superoxide dismutase-1 enzyme. Over 100 such mutations have been identified in humans. The mutations are characterized as “gain-of-function” mutations, because they are dominant to wild-type alleles. Moreover, at least some of the mutations do not appear to affect the enzyme activity.

Systemic delivery of potentially therapeutic neuroprotective factors has been disappointing. Recently, delivery of viral vector-encoded IGF-1 to peripheral muscle has demonstrated beneficial effects on disease progression in a mouse model. This has been attributed to retrograde transport of viral particles. Intrathecal administration of IGF-1 into the lumbar spinal cord has also been found to be efficacious in mouse models, improving motor performance, delaying the onset of diseases, and extending survival.

There is a continuing need in the art for methods to treat ALS in patients.

According to one embodiment of the invention, a patient with Amyotrophic Lateral Sclerosis (ALS) is treated by administering an insulin-like growth factor-1 (IGF-1). The administration to the patient is performed via intraventricular delivery to the brain. An amount of the IGF-1 that is sufficient to reduce ALS disease progression is administered. In a first aspect, the present invention therefore provides for a method for the treatment and/or prevention of ALS in a patient, said method comprising the administration of an IGF-1, to the brain of the patient via intraventricular delivery. In a related aspect, the invention provides for the use of an IGF-1, for the manufacture of a medicament for the treatment and/or prevention of ALS in a patient, wherein the treatment or prevention comprises the intraventricular administration of an IGF-1 to the brain.

Another aspect of the invention is a kit for treating a patient with Amyotrophic Lateral Sclerosis. The kit comprises an insulin-like growth factor-1 (IGF-1), and a catheter for delivery of said insulin-like growth factor-1 (IGF-1) to one or more of the patient's brain ventricles.

Yet another aspect of the invention is a further kit for treating a patient with Amyotrophic Lateral Sclerosis. The kit comprises an insulin-like growth factor-1 (IGF-1), and a pump for delivery of said insulin-like growth factor-1 (IGF-1) to one or more of the patient's brain ventricles. Any of the kits of the present invention may comprise both a catheter and a pump. Any catheter or pump that is used in the present invention may be specifically designed or adapted for the intraventricular administration of a medicament to the brain.

These and other embodiments which will be apparent to those of skill in the art upon reading the specification provide the art with methods and kits for treatment of Amyotrophic Lateral Sclerosis.

### BRIEF DESCRIPTION OF THE DRAWINGS

FIG. 1 shows a cross section view of the human brain with the ventricles indicated.

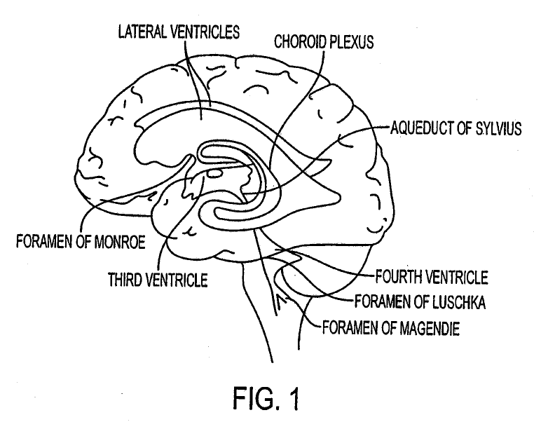

FIGS. 2A and 2B show lateral and superior views, respectively, of the ventricles.

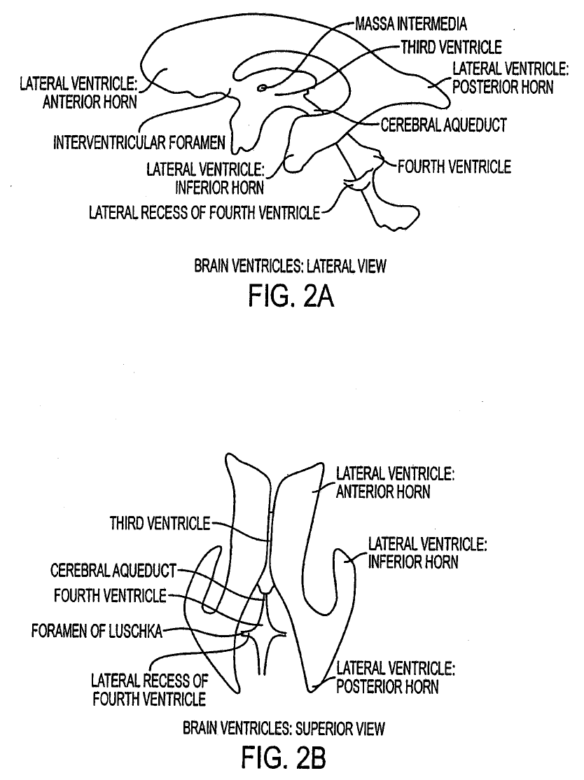

FIG. 3 shows injection into the ventricles.

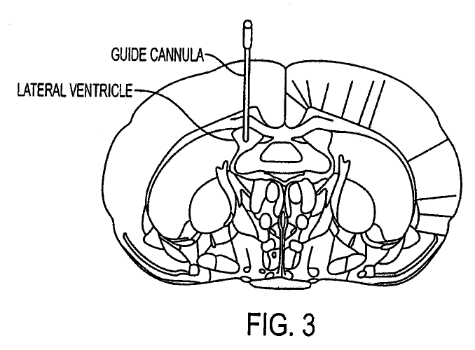

FIG. 4 shows the flow of CSF through the ventricles with eventual absorption through arachnoid villi into the superior sagittal sinus and the blood circulation.

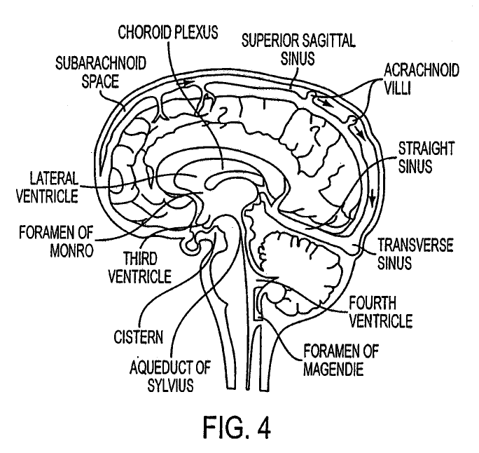

### DETAILED DESCRIPTION OF THE INVENTION

The practice of the present invention will employ, unless otherwise indicated, conventional techniques of immunology, molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

As used in the specification and claims, the singular forms “a,” “an” and “the” include plural references unless the context clearly dictates otherwise. For example, the term “a cell” includes a plurality of cells, including mixtures thereof.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and excluding substantial method steps for administering the compositions or medicaments in accordance with this invention. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about.” It also is to be understood, although not always explicitly stated, that the reagents described herein are merely exemplary and that equivalents of such that are known in the art may also be used.

The terms “therapeutic,” “therapeutically effective amount,” and their cognates refer to that amount of a substance, e.g., of a protein, e.g., of an IGF-1, that results in prevention or delay of onset, or amelioration, of one or more symptoms of a disease, e.g., ALS, in a subject, or an attainment of a desired biological outcome, such as correction of neuropathology, e.g., cellular pathology associated with a motor neuronal disease such as ALS. The term “therapeutic correction” refers to that degree of correction which results in prevention or delay of onset, or amelioration, of one or more symptoms in a subject. The effective amount can be determined by known empirical methods.

A “composition” or “medicament” is also intended to encompass a combination of an active agent, e.g., IGF-1, and a carrier or other material, e.g., a compound or composition, which is inert (for example, a detectable agent or label) or active, such as an adjuvant, diluent, binder, stabilizer, buffer, salt, lipophilic solvent, preservative, adjuvant or the like, or a mixture of two or more of these substances. Carriers are preferably pharmaceutically acceptable. They may include pharmaceutical excipients and additives, proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

The term carrier also includes a buffer or a pH adjusting agent or a composition containing the same; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid, Tris, tromethamine hydrochloride, or phosphate buffers. Additional carriers include polymeric excipients/additives such as polyvinylpyrrolidones, ficolls (a polymeric sugar), dextrates (e.g., cyclodextrins, such as 2-hydroxypropyl.-quadrature.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions and medicaments which are manufactured and/or used in accordance with the present invention and which include an IGF-1 can include stabilizers and preservatives and any of the above noted carriers with the additional proviso that they be acceptable for use in vivo. For examples of carriers, stabilizers and adjuvants, see Martin REMINGTON'S PHARM. SCI., 15th Ed. (Mack Publ. Co., Easton (1975) and Williams & Williams, (1995), and in the “PHYSICIAN'S DESK REFERENCE”, 52^{nd} ed., Medical Economics, Montvale, N.J. (1998).

A “subject,” “individual” or “patient” is used interchangeably herein, which refers to a vertebrate, preferably a mammal, more preferably a human. Mammals include, but are not limited to, mice, rats, monkeys, humans, farm animals, sport animals, and pets.

As used herein, the term “modulate” means to vary the amount or intensity of an effect or outcome, e.g., to enhance, augment, diminish or reduce.

As used herein the term “ameliorate” is synonymous with “alleviate” and means to reduce or lighten. For example, one may ameliorate the symptoms of a disease or disorder by making them more bearable.

For identification of structures in the human brain, see, e.g., The Human Brain: Surface, Three-Dimensional Sectional Anatomy With MRI, and Blood Supply, 2nd ed., eds. Deuteron et al., Springer Vela, 1999; Atlas of the Human Brain, eds. Mai et al., Academic Press; 1997; and Co-Planar Stereotaxic Atlas of the Human Brain: 3-Dimensional Proportional System: An Approach to Cerebral Imaging, eds. Tamarack et al., Thyme Medical Pub., 1988. For identification of structures in the mouse brain, see, e.g., The Mouse Brain in Stereotaxic Coordinates, 2nd ed., Academic Press, 2000.

Intraventricular delivery of IGF-1 to subjects with ALS leads to improved status of the central nervous system. This is particularly true when the delivery rate is slow, relative to a bolus delivery. Particularly useful proteins for treating ALS are the A and B isoforms of insulin-like grown factor (IGF-1), shown in SEQ ID NO: 1 and SEQ ID NO: 2. Other isoforms may also be used. Distinct proteins which may be used, alone or in combination with each other in accordance with the present invention include IGF-1, VEGF, and GDNF.

The insulin-like growth factor (IGF-1) gene has a complex structure, which is well-known in the art. It has at least two alternatively spliced mRNA products arising from the gene transcript. There is a 153 amino acid peptide, known by several names including IGF-1A or IGF-1Ea, and a 195 amino acid peptide, known by several names including IGF-1B or IGF-1Eb. The mature form of IGF-1 is a 70 amino acid polypeptide. Both IGF-1Ea and IGF-1Eb contain the 70 amino acid mature peptide, but differ in the sequence and length of their carboxyl-terminal extensions. The peptide sequences of IGF-1Ea and IGF-1Eb are represented by SEQ ID NOS: 1 and 2, respectively. The genomic and functional cDNAs of human IGF-1, as well as additional information regarding the IGF-1 gene and its products, are available at Unigene Accession No. NM_{—}00618. Allelic variants may differ by a single or a small number of amino acid residues, typically less than 5, less than 4, less than 3 residues.

Although a particular amino acid sequence for IGF-1 is shown in each of SEQ ID NO: 1 and SEQ ID NO: 2, variants of those sequences which retain activity, e.g., normal variants in the human population, can be used as well. Typically these normal variants differ by just one or two residues from the sequence shown in SEQ ID NO: 1 or SEQ ID NO: 2. The variants to be used should be at least 95%, 96%, 97%, 98%, or 99% identical to SEQ ID NO: 1 or SEQ ID NO: 2. Variants which are associated with disease or reduced activity should not be used. Precursor forms (pre-, pro-, or prepro-forms) may also be administered for in vivo processing. In one embodiment, the IGF-1 protein is a recombinant form of the protein that is produced using methods that are well-known in the art. In another embodiment, it is a recombinant human IGF-1 protein.

Without being limited as to theory, IGF-1 is a therapeutic protein for the treatment of ALS due to its many actions at different levels of neuraxis (see Dore et al., Trends Neurosci, 1997, 20:326-331). In the brain: It is thought to reduce both neuronal and glial apoptosis, protect neurons against toxicity induced by iron, colchicine, calcium destabilizers, peroxides, and cytokines. It also is thought to modulate the release of neurotransmitters acetylcholine and glutamate. It is also thought to induce the expression of neurofilament, tublin, and myelin basic protein. In the spinal cord: IGF-1 is thought to modulate ChAT activity and attenuate loss of cholinergic phenotype, enhance motor neuron sprouting, increase myelination, inhibit demyelination, stimulate motor neuron proliferation and differentiation from precursor cells, and promote Schwann cell division, maturation, and growth. In the muscle: IGF-1 is thought to induce acetylcholine receptor cluster formation at the neuromuscular junction and increase neuromuscular function and muscle strength.

Kits according to the present invention are assemblages of separate components. While they can be packaged in a single container, they can be subpackaged separately. Even a single container can be divided into compartments. Typically a set of instructions will accompany the kit and provide instructions for delivering the IGF-1, intraventricularly. The instructions may be in printed form, in electronic form, as an instructional video or DVD, on a compact disc, on a floppy disc, on the internet with an address provided in the package, or a combination of these means. Other components, such as diluents, buffers, solvents, tape, screws, and maintenance tools can be provided in addition to the IGF-1, one or more cannulae or catheters, and/or a pump.

The populations treated by the methods of the invention include, but are not limited to, patients having or at risk for developing ALS.

An IGF-1 protein can be incorporated into a pharmaceutical composition useful to treat, e.g., inhibit, attenuate, prevent, or ameliorate, a symptom caused by ALS. The pharmaceutical composition will be administered to a subject suffering from ALS or someone who is at risk of developing ALS. The compositions should contain a therapeutic or prophylactic amount of the protein in a pharmaceutically-acceptable carrier. The pharmaceutical carrier can be any compatible, non-toxic substance suitable to deliver the polypeptides to the patient. Sterile water, alcohol, fats, and waxes may be used as the carrier. Pharmaceutically-acceptable adjuvants, buffering agents, dispersing agents, and the like, may also be incorporated into the pharmaceutical compositions. The carrier can be combined with the protein in any form suitable for administration by intraventricular injection or infusion (which form is also possibly suitable for intravenous or intrathecal administration) or otherwise. Suitable carriers include, for example, physiological saline, bacteriostatic water, Cremophor EL.TM. (BASF, Parsippany, N.J.) or phosphate buffered saline (PBS), other saline solutions, dextrose solutions, glycerol solutions, water and oils emulsions such as those made with oils of petroleum, animal, vegetable, or synthetic origin (peanut oil, soybean oil, mineral oil, or sesame oil). An artificial CSF can be used as a carrier. The carrier will preferably be sterile and free of pyrogens. The concentration of the protein in the pharmaceutical composition can vary widely, i.e., from at least about 0.01% by weight, to 0.1% by weight, to about 1% weight, to as much as 20% by weight or more of the total composition.

For intraventricular administration of IGF-1, VEGF or GDNF, the composition must be sterile and should be fluid. It must be stable under the conditions of manufacture and storage and must be preserved against the contaminating action of microorganisms such as bacteria and fungi. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents in the composition, for example, sugars, polyalcohols such as mannitol, sorbitol, and sodium chloride.

IGF-1, VEGF or GDNF protein may be infused into any one of the brain's ventricles. The ventricles are filled with cerebrospinal fluid (CSF). CSF is a clear fluid that fills the ventricles, is present in the subarachnoid space, and surrounds the brain and spinal cord. CSF is produced by the choroid plexuses and via the weeping or transmission of tissue fluid by the brain into the ventricles. The choroid plexus is a structure lining the floor of the lateral ventricle and the roof of the third and fourth ventricles. Certain studies have indicated that these structures are capable of producing 400-600 ccs of fluid per day consistent with an amount to fill the central nervous system spaces four times in a day. In adults, the volume of this fluid has been calculated to be from 125 to 150 ml (4-5 oz). The CSF is in continuous formation, circulation and absorption. Certain studies have indicated that approximately 430 to 450 ml (nearly 2 cups) of CSF may be produced every day. Certain calculations estimate that production equals approximately 0.35 ml per minute in adults and 0.15 per minute in infants. The choroid plexuses of the lateral ventricles produce the majority of CSF. It flows through the foramina of Monro into the third ventricle where it is added to by production from the third ventricle and continues down through the aqueduct of Sylvius to the fourth ventricle. The fourth ventricle adds more CSF; the fluid then travels into the subarachnoid space through the foramina of Magendie and Luschka. It then circulates throughout the base of the brain, down around the spinal cord and upward over the cerebral hemispheres. The CSF empties into the blood via the arachnoid villi and intracranial vascular sinuses, thereby potentially delivering a protein infused into the ventricles to not only the central nervous system but also to the bloodstream.

Dosage of the IGF-1 protein, may vary somewhat from individual to individual, depending on the particular protein and its specific in vivo activity, the route of administration, the medical condition, age, weight or sex of the patient, the patient's sensitivities to the IGF-1 or other neurotrophic growth factor or components of vehicle, and other factors which the attending physician will be capable of readily taking into account.

The rate of administration is such that the administration of a single dose may be administered as a bolus. A single dose may also be infused over about 1-5 minutes, about 5-10 minutes, about 10-30 minutes, about 30-60 minutes, about 1-4 hours, or consumes more than four, five, six, seven, or eight hours. It may take more than 1 minute, more than 2 minutes, more than 5 minutes, more than 10 minutes, more than 20 minutes, more than 30 minutes, more than 1 hour, more than 2 hours, or more than 3 hours. Applicants have observed that, while bolus intraventricular administration of a protein may be effective, slow infusion is very effective. While applicants do not wish to be bound by any particular theory of operation, it is believed that the slow infusion is effective due to the turn-over of the cerebrospinal fluid (CSF). While estimates and calculations in the literature vary, the cerebrospinal fluid in humans is believed to turn over within about 4, 5, 6, 7, or 8 hours. The slow infusion of the invention should be metered so that it is about equal to or greater than the turn-over time of the CSF. Turn-over time may depend on the species, size, and age of the subject but may be determined using methods known in the art. Infusion may also be continuous over a period of one or more days. The patient may be treated once, twice, or three or more times a month, e.g., weekly, e.g., every two weeks. Infusions may be repeated over the course of a subject's life.

The CSF empties into the blood via the arachnoid villi and intracranial vascular sinuses, thereby delivering the infused protein to the lower motor neurons and skeletal muscles. The reduction in symptoms can be dramatic and may include reduction in one of the following: a reduction in the subject's weakness of limbs, a reduction in the slurring of the subject's speech, a reduction in the subject's difficulty swallowing, and a reduction in the subject's difficulty breathing. The treated subject's survival time may increase relative to a non-treated subject with ALS.

Reductions of greater that 10%, 20%, 30%, 40%, 50%, 60%, 70%, 80%, 90% can be achieved. The reduction achieved is not necessarily uniform from patient to patient or even from symptom to symptom within a single patient.

In one embodiment, administration an IGF-1, is accomplished by infusion of the protein into one or both of the lateral ventricles of a subject or patient. By infusing into the lateral ventricles, the protein is delivered to the site in the brain in which the greatest amount of CSF is produced. The protein may also be infused into more than one ventricle of the brain. Treatment may consist of a single infusion per target site, or may be repeated. Multiple infusion/injection sites can be used. For example, the ventricles into which the protein is administered may include the lateral ventricles and the fourth ventricle. In some embodiments, in addition to the first administration site, a composition containing the IGF-1 protein is administered to another site which can be contralateral or ipsilateral to the first administration site. Injections/infusions can be single or multiple, unilateral or bilateral.

To deliver the solution or other composition containing the protein specifically to a particular region of the central nervous system, such as to a particular ventricle, e.g., to the lateral ventricles or to the fourth ventricle of the brain, it may be administered by stereotaxic microinjection. For example, on the day of surgery, patients will have the stereotaxic frame base fixed in place (screwed into the skull). The brain with stereotaxic frame base (MRI-compatible with fiduciary markings) will be imaged using high resolution MRI. The MRI images will then be transferred to a computer that runs stereotaxic software. A series of coronal, sagittal and axial images will be used to determine the target site of vector injection, and trajectory. The software directly translates the trajectory into 3-dimensional coordinates appropriate for the stereotaxic frame. Burr holes are drilled above the entry site and the stereotaxic apparatus localized with the needle implanted at the given depth. The protein solution in a pharmaceutically acceptable carrier will then be injected. Additional routes of administration may be used, e.g., superficial cortical application under direct visualization, or other non-stereotaxic application.

A pump is one means to slowly infuse a therapeutic protein into the ventricles of a subject. Such pumps are commercially available, for example, from Alzet (Cupertino, Calif.) or Medtronic (Minneapolis, Minn.). The pump may optionally be implantable. Another convenient way to administer the protein, is to use a cannula or a catheter. The cannula or catheter may be used for multiple administrations separated in time. Cannulae and catheters can be implanted stereotaxically. It is contemplated that multiple administrations over time will be used to treat the typical patient with ALS. Catheters and pumps can be used separately or in combination.

The subject invention provides methods to modulate, correct, or augment motor function in a subject afflicted with motor neuronal damage. For the purpose of illustration only, the subject may suffer from one or more of symptoms of amyotrophic lateral sclerosis (ALS), such as exertional/rest dyspnea, orthopnea, poor cough, constipation, low voice volume, poor quality sleep, morning headache, daytime sleepiness, apneas, choking spells, noisy breathing, coughing with eating, clumsiness, twitching, cramping, weakness, slurring of speech, difficulty with speech and swallowing, and pathological laughing or crying.

The ability to organize and execute complex motor acts depends on signals from the motor areas in the cerebral cortex, i.e., the motor cortex. Cortical motor commands descend in two tracts. The corticobular fibers control the motor nuclei in the brain stem that move facial muscles and the corticospinal fibers control the spinal motor neurons that innervate the trunk and limb muscles. The cerebral cortex also indirectly influences spinal motor activity by acting on the descending brain stem pathways. The primary motor cortex lies along the precentral gyrus in Broadmann's area (4). The axons of the cortical neurons that project to the spinal cord run together in the corticospinal tract, a massive bundle of fibers containing about 1 million axons. About a third of these originate from the precentral gyrus of the frontal lobe. Another third originate from area 6. The remainder originates in areas 3, 2, and 1 in the somatic sensory cortex and regulate transmission of afferent input through the dorsal horn.

The corticospinal fibers run together with corticobulbar fibers through the posterior limb of the internal capsule to reach the ventral portion of the midbrain. They separate in the pons into small bundles of fibers that course between the pontine nuclei. They regroup in the medulla to form the medullary pyramid. About three-quarters of the corticospinal fibers cross the midline in the pyramidal decussation at the junction of the medulla and spinal cord. The crossed fibers descend in the dorsal part of the lateral columns (dorsolateral column) of the spinal cord, forming the lateral corticospinal tract. The uncrossed fibers descend in the ventral columns as the ventral corticospinal tract.

The lateral and ventral divisions of the corticospinal tract terminate in about the same regions of spinal gray matter as the lateral and medial systems of the brain stem. The lateral corticospinal tract projects primarily to motor nuclei in the lateral part of the ventral horn and to interneurons in the intermediate zone. The ventral corticospinal tract projects bilaterally to the ventromedial cell column and to adjoining portions of the intermediate zone that contain the motor neurons that innervate axial muscles. Deep within the cerebellum is grey matter called the deep cerebellar nuclei termed the medial (fastigial) nucleus, the interposed (interpositus) nucleus and the lateral (dentate) nucleus. As used herein, the term “deep cerebellar nuclei” collectively refers to these three regions.

If desired, the human brain structure can be correlated to similar structures in the brain of another mammal. For example, most mammals, including humans and rodents, show a similar topographical organization of the entorhinal-hippocampus projections, with neurons in the lateral part of both the lateral and medial entorhinal cortex projecting to the dorsal part or septal pole of the hippocampus, whereas the projection to the ventral hippocampus originates primarily from neurons in medial parts of the entorhinal cortex (Principles of Neural Science, 4th ed., eds Kandel et al., McGraw-Hill, 1991; The Rat Nervous System, 2nd ed., ed. Paxinos, Academic Press, 1995). Furthermore, layer II cells of the entorhinal cortex project to the dentate gyrus, and they terminate in the outer two-thirds of the molecular layer of the dentate gyrus. The axons from layer III cells project bilaterally to the cornu ammonis areas CA1 and CA3 of the hippocampus, terminating in the stratum lacunose molecular layer.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

### Animal Models

Several transgenic animal models of adult onset motor neuron diseases have been developed which employ human ALS-associated SOD1 mutations. These models are useful for preclinical therapeutic studies. One popular and established model employs the SOD1^{G93A} allele as a transgene in mice. Gurney, M E, et al., Science, 264: 1772-1775, 1994; and Tu, P. H. et al, Proc. Natl. Acad. Sci. USA 93: 3155-3160 (1996).

This allele was originally found in some human patients with familial ALS. Li, B. et al., Brain Res. Mol. Brain Res. 111, 155-164, 2003. These mice have been found to share the phenotypic features of ALS. Such mice are available from the Jackson Laboratory, Bar Harbor, Me.

### EXAMPLE 2

### Intraventricular Infusion of rhIGF-1 in the SOD1^{G93A} mouse

Goal: To determine what effect intraventricular infusion of recombinant human IGF-1 (rhIGF-1) has on ALS disease progression.

Methods: SOD1^{G93A} mice are stereotaxically implanted with an indwelling guide cannula between 12 and 13 weeks of age. At 14 weeks of age mice are infused with rhIGF-1 (n=5) over a 24 h period for four straight days using an infusion probe (fits inside the guide cannula) which is connected to a pump. Lyophilized rhIGF-1 is dissolved in artificial cerebral spinal fluid (aCSF) prior to infusion. Mice are sacrificed 3 days post infusion. At sacrifice mice are overdosed with euthasol (>150 mg/kg) and then perfused with PBS or 4% parformaldehyde. Motor neurons are examined histologically. Serum levels of IGF-1 are assessed periodically during the in-life phase of the experiment. ALS disease progression is evaluated over time.

### EXAMPLE 3

### Intraventricular Delivery of rhIGF-1 in SOD1^{G93A} Mice

Goal: to determine lowest efficacious dose over a 6 hour infusion period.

Methods: SOD1^{G93A} mice are stereotaxically implanted with an indwelling guide cannula between 12 and 13 weeks of age. At 14 weeks of age mice are infused over a 6 hour period with rhIGF-1 or aCSF (artificial cerebral spinal fluid). Two mice from each dose level are perfused with 4% parformaldehyde immediately following the 6 h infusion to assess protein distribution in the brain (blood is collected from these mice to determine serum IGF-1 levels). The remaining mice from each group are sacrificed 1 week post infusion. Motor neurons are examined histologically. Serum levels of are assessed periodically during the in-life phase of the experiment. ALS disease progress is evaluated over time.

We claim:

1. A method of treating a patient with Amyotrophic Lateral Sclerosis (ALS), comprising administering an insulin-like growth factor-1 (IGF-1), to the patient via intraventricular delivery to the brain in an amount sufficient to reduce ALS disease progression.

2. The method of claim 1 wherein the amount administered is sufficient to increase survival time.

3. The method of claim 1 wherein the amount administered is sufficient to reduce weakness of limbs.

4. The method of claim 1 wherein the amount administered is sufficient to reduce slurring of speech.

5. The method of claim 1 wherein the amount administered is sufficient to reduce difficulty swallowing.

6. The method of claim 1 wherein the amount administered is sufficient to reduce difficulty breathing.

7. The method of claim 1 wherein the amount administered is sufficient to reduce sleep apnea.

8. The method of any one of claims 1-7 wherein the method comprises the administration of an insulin-like growth factor-1 (IGF-1), and said IGF-1 is preferably a human insulin-like growth factor-1 (IGF-1).

9. The method of any one of claims 1-8 wherein the intraventricular delivery to the brain is performed by injecting the insulin-like growth factor-1 (IGF-1) into a lateral ventricle of the patient.

10. The method of claim 1 wherein the intraventricular delivery to the brain is performed by injecting the insulin-like growth factor-1 (IGF-1) into the lateral ventricles and the fourth ventricle of the patient.

11. The method of any preceding claim wherein the insulin-like growth factor-1 (IGF-1) shares at least 95% amino acid sequence identify with an insulin-like growth factor-1 (IGF-1) as shown in SEQ ID NO: 1 or 2.

12. The method of claim 11 wherein the insulin-like growth factor-1 (IGF-1) shares at least 96% amino acid sequence identify with an insulin-like growth factor-1 (IGF-1) as shown in SEQ ID NO: 1 or 2.

13. The method of claim 12 wherein the insulin-like growth factor-1 (IGF-1) shares at least 97% amino acid sequence identify with an insulin-like growth factor-1 (IGF-1) as shown in SEQ ID NO: 1 or 2.

14. The method of claim 13 wherein the insulin-like growth factor-1 (IGF-1) shares at least 98% amino acid sequence identify with an insulin-like growth factor-1 (IGF-1) as shown in SEQ ID NO: 1 or 2.

15. The method of claim 14 wherein the insulin-like growth factor-1 (IGF-1) shares at least 99% amino acid sequence identify with an insulin-like growth factor-1 (IGF-1) as shown in SEQ ID NO: 1 or 2.

16. The method of claim 1 wherein the insulin-like growth factor-1 (IGF-1) has a sequence as shown in SEQ ID NO: 1.

17. The method of claim 1 wherein the insulin-like growth factor-1 (IGF-1) has a sequence as shown in SEQ ID NO: 2.

18. The method of any preceding claim wherein the step of administering comprises a plurality of infusions.

19. The method of claim 1 wherein the step of administering is performed at a rate such that the administration of a single dose consumes more than four hours.

20. The method of claim 1 wherein the step of administering is performed at a rate such that the administration of a single dose consumes more than five hours.

21. The method of claim 1 wherein the step of administering is performed at a rate such that the administration of a single dose consumes more than six hours.

22. The method of claim 1 wherein the step of administering is performed at a rate such that the administration of a single dose consumes more than seven hours.

23. The method of claim 1 wherein the step of administering is performed at a rate such that the administration of a single dose consumes more than eight hours.
